(19) United States
(12) Patent Application Publication
(10) Pub. No.: US 2022/0395455 A1
(43) Pub. Date: Dec. 15, 2022

(54) METHODS AND PROCESSES FOR ENHANCING THE AESTHETIC APPEARANCE OF A FACE

(71) Applicants: SEBASTIAN COTOFANA, Rochester, MN (US); CLAUDIA ANDREA HERNANDEZ, Medellin (CO)

(72) Inventors: SEBASTIAN COTOFANA, Rochester, MN (US); CLAUDIA ANDREA HERNANDEZ, Medellin (CO)

(21) Appl. No.: 17/344,707

(22) Filed: Jun. 10, 2021

(51) Int. Cl. A61K 9/00; A61L 27/20; A61L 27/54; A61L 27/12; A61M 5/32

(52) U.S. Cl. CPC A61K 9/0019; A61L 27/20; A61L 27/54; A61L 27/12; A61M 5/32; A61L 2430/34; A61L 2300/236; A61M 2210/0606

(57) ABSTRACT

The present patent document describes methods for enhancing the aesthetic appearance of a human face by injecting soft tissue filler material in the temporal region. The soft tissue filler material is injected into a subdermal plane; preferably from an injection site about 1 cm anterior to the apex of the tragus. A single injection may be performed on either side of the face.

### FIELD OF THE INVENTION

This patent document relates generally to methods and process for enhancing the aesthetic appearance of a human face. More particularly, the subject matter of this patent document relates to enhancing the aesthetic appearance of a human face by injecting soft tissue volumizing substances into the temporal region to increase the volume of the temporal soft tissues, reduce the volume of the rest of the face and increase the contour between face and neck.

### BACKGROUND

In 2019, a total of 784.185 soft tissue filler injections of hyaluronic acid (HA) and calcium hydroxylapatite (CaHA) products were performed in the United States according to the annual statistics report of The Aesthetic Society. This represents an increase by 18.7% when compared to the usage of the same products in 2015 and is reflective of an increased desire for esthetic enhancement. To this end, there is an ever-increasing need for new, better and safer methods of enhancing the aesthetic appearance human face.

### SUMMARY

Methods and processes for enhancing the appearance of a human face are provided. In preferred embodiments, the method of enhancing the aesthetic appearance of a human face comprises first inserting an injection device into the subdermal plane of the human face. Once the injection device is inserted into the subdermal plane, the injection device is advanced in the subdermal plane to an injection location of the temporal region. The injection location is most ideally located in the horizontal plane between 0.5 cm and 1.5 cm anterior to an apex of a tragus. Product deposition is located in the temple between 4 cm and 6 cm above (cranially) to the dermal access site. After the injection device reaches the deposition location (temporal region), a product deposit with an amount between 0.5 cc and 1.5 cc of soft tissue volumizing material is administered in the subdermal plane of the temple.

In preferred embodiments, the product deposition location is about 1 cm anterior to the apex of the tragus in the horizontal plane, and about 5 cm superior (cranial) to the horizontal plane running through the apex of the tragus.

Although the injection device can be inserted and advanced to the injection location in many different ways, preferably, the injection device is inserted in an insertion location about 1 cm anterior to an apex of a tragus and advance in a superior direction to the product deposition location.

In various different embodiments, different injection devices may be used. Preferably the injection device to create the dermal access is a sharp-tip needle and the injection device to advance in the subdermal plane after the dermal access was established is a blunt-tip cannula. Even more preferably the needle is a 21G needle and the cannula is a 22G 50 mm to 70-mm blunt-tip cannula.

While it is not necessary to insert the entire cannula, preferably the entire length of the cannula is advanced in the subdermal plane to reach the product deposition location in the temporal region. Preferably, once the cannula is introduced into the temporal soft tissues, the long axis of the cannula is lifted towards the underside of the skin to increase safety by confirming the subdermal injection location.

Once the product deposition location in the temple is reached, the soft tissue filler is preferably injected slowly. The soft tissue filler may be injected at a rate between 0.1 cc per second and 0.2 cc per second.

In order to reduce retrograde flow of the soft tissue filler during the injection process, pressure may be applied on an exterior of the skin superficial to the cannula to compress an injection canal created by the cannula.

As may be appreciated, there are many soft tissue fillers and the method disclosed herein are generally not restricted to any of them. However, preferably the soft tissue filler is an HA-based soft tissue filler or a CaHA material. In preferred embodiments, between 0.75 cc and 1.0 cc of soft tissue filler material is injected. However, in other embodiments, other amounts of soft tissue filler may be used.

While in rare cases, the injection may be done on only one side of the face, typically, a single injection in the temporal region is done on each side of the human face.

### BRIEF DESCRIPTION OF THE DRAWINGS

These and other features, aspects and advantages are described below with reference to the drawings, which are intended to illustrate but not to limit the invention. In the drawings, like reference characters denote corresponding features consistently throughout similar embodiments.

FIG. 1 is a profile view of a human head.

### DETAILED DESCRIPTION

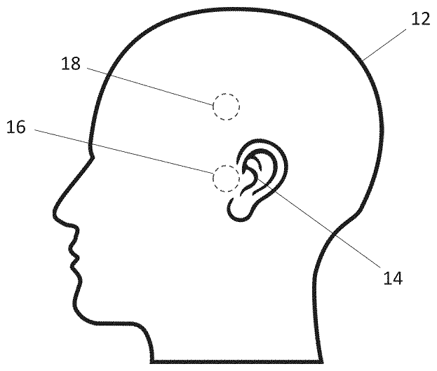

Unique and inventive methods for improving the aesthetic appearance of a human face are disclosed herein. FIG. 1 is a profile view of a human head 12. In general, the aesthetic appearance of the face is enhanced by depositing a soft tissue filler material into the subdermal plane of the temporal region 18. As shown herein, such an injection creates a measurable volumizing effect at the product deposition location of the temple and a lifting of the facial features, resulting in a measurable improvement in appearance.

In the methods disclosed herein, any type of soft tissue filler material, also referred to as dermal fillers or volumizing fillers, may be used. As just a few examples. Calcium Hydroxylapatite (CaHA), Hyaluronic Acid (HA), Polyalkylimide, Polylactic acid. Polymethyl-methacrylate microspheres (PMMA), autologous fat or even implants may be used. However, in preferred embodiments, either CaHA or HA are used.

In particular, soft tissue filler with higher G-prime properties are preferred. In this case, CaHA is preferred over HA. However, as already stated, any soft tissue filler can be used. Generally, any product with high G-prime properties favoring greater lifting effects may be used.

An injection device is used to inject the soft tissue filler material. Any type of injection device or method may be used. In some embodiments, the dermal filler may be injected with just a needle. In preferred embodiments, a needle and a cannula are used. The needle is used to create an entry point for the cannula. The cannula is then inserted into the puncture site and maneuvered along in the subdermal plane to the deposition location in the temple where the dermal filler is desired.

In preferred embodiments, a 21G needle or larger is used in combination with a 22G 50 mm to 70 mm cannula. As just one example, such an injection device is available from Feeltech CO Ltd. In other embodiments, other needle sizes and other cannula sizes may be used.

In embodiments that use a cannula, a blunt-tip cannula is preferably used. Other types of cannulas may be used without departing from the scope of the inventive methods described herein but a blunt-tip cannula is preferably used.

The soft tissue filler is administered into the superficial fat compartments (=Layer 2) utilizing the injection device. The posterior (parietal) branch and the anterior (frontal) branch of the superficial temporal artery are located inside the superficial temporal fascia (=Layer 3); this indicates that the product is injected superficial to the arteries (and the accompanying veins). To ensure subdermal (=Layer 2) product placement, the cannula is preferably lifted against the underside of the skin as it is advanced to prevent the tip from diving into deeper planes when performing this technique.

If product is accidently introduced into the superficial temporal artery complications may result including livedo and partial tissue loss of large areas of the ipsilateral scalp and the ipsilateral oral mucosa without injection-related visual compromise. Utilizing a 22G cannula additionally decreases the risk for arterial puncture and therefore direct intra-arterial product application as a previous study showed that the force needed to penetrate an artery is statistically significantly reduced compared to smaller sized cannulas such as a 25G or 27G cannula.

The amount of dermal filler that is used can vary based on the size of the patients' face, sex, mean body index, or desired effect. However, in preferred embodiments, between 0.5 cc and 1.5 cc of soft tissue volumizing material is used. In more preferred embodiments, between 0.75 cc and 1.0 cc of volumizing material is used. Even more preferably, about 0.75 cc of CaHA soft tissue filler is used or about 1.0 cc of HA-based soft tissue filler is used. As used herein with respect to the volume of dermal filler, the term “about” means±0.5 cc.

As one skilled in the art will appreciate, lots of different dermal fillers are available from numerous different manufactures and various different dermal fillers may be used without departing from the scope and spirit of the inventive methods disclosed herein. As just one example of an HA-based soft tissue filler material that may be used, Belotero® Volume is manufactured by Merz Pharma GmbH in Frankfurt Germany. As just one example of a CaHA material. Radiesse® is manufactured by Merz Pharma North America based in Raleigh N.C. United States.

The soft tissue filler material is placed in the subdermal layer in the temporal region 18. The preferred product deposition location 18 for the dermal filler is about 1 cm anterior to the apex of the tragus 14 in the horizontal plane and about 5 cm superior (cranial) to the horizontal plane of the apex of the tragus 14. As used herein when referring to the injection location, the term “about” means±0.3 cm.

When discussing the deposition location 18 of the dermal filler, the location of the tip of the injection device during administration is described. As is known, the dermal filler will distribute within the subdermal plane of the temple as it is injected. However, in the embodiments herein, the dermal filler is preferably localized as much as possible using a bolus technique. A bolus technique simply means that the injection device is held as still as possible while the dermal filler is administered.

In other embodiments, other injection techniques may be used without departing from the scope of the inventive methods herein; however, a bolus technique with no fanning is preferred for the injection technique of the embodiments herein.

In the embodiments described herein, the ideal product deposition location 18 is 1 cm anterior of the apex of the tragus 14 and 5 cm superior (cranial) to the horizontal plane of the apex of the tragus 14. However, the product deposition location 18 can vary slightly without departing from the inventive methods herein. In preferred embodiments, the product deposition location 18 can be anywhere in a 2 cm diameter area that has its center 1 cm anterior of the apex of the tragus and 5 cm superior to the horizontal plane of the apex of the tragus 14. To this end, the product deposition location 18 can be anywhere between 0 cm and 2 cm anterior of the apex of the tragus and between 4.0 cm and 6.0 cm superior to the horizontal plane through the apex of the tragus 14. Even more preferably, the product deposition location 18 is between 0.5 and 1.5 cm anterior to the apex of the tragus 14 and between 4.5 cm and 5.5 cm superior to the horizontal plane through the apex of the tragus 14.

In preferred embodiments, the product deposition location 18 is always hidden behind the hairline. Keeping the injection location 18 underneath the hairline reduces visibility.

In order to reach the product deposition location 18, the injection device must be inserted into the subdermal plane. Prior to insertion, although not required, it is recommended that the dermal access location 16 is disinfected with a topical disinfectant such as alcohol or other topical disinfectant.

The dermal access location 16 of the injection device is preferably about 1 cm anterior of the apex of the tragus 14. The dermal access location 16 is not as critical as the product deposition location 18 and in some embodiments, the dermal access location 16 may be located in other locations. However, the dermal access location 16 is preferably 1 cm anterior of the apex of the tragus 14.

In some embodiments, the dermal access location 16 can be between 0.5 cm and 1.5 cm anterior of the apex of the tragus in the horizontal plane 14. As may be appreciated, the dermal access location 16 cannot be done much closer to the apex of the tragus 14 but could be moved more in the anterior direction. In such embodiments, the dermal access location 16 could be up to 10 cm anterior to the apex of the tragus 14 and then the injection device would be angled posterior during insertion to reach the product deposition location 18 in the temporal region behind the hairline. In other embodiments, other dermal access locations 16 may be possible as long as the product deposition location 18 is successfully reached.

In preferred embodiments, the dermal access location 16 is done directly anterior of the apex of the tragus 14 and then the injection device is advanced fully to reach the product deposition location 18. However, in other embodiments, the dermal access location 16 may vary in either the superior or inferior direction. For example, the dermal access location 16 may vary in either the superior direction anywhere from directly anterior to the apex of the tragus 14 all the way up to the product deposition location 18 if the injection device is not fully inserted or a shorter injection device is used. Moreover, the dermal access location 16 may vary in the inferior direction if a longer injection device is used.

Preferably, the dermal access location 16 is directly anterior to the apex of the tragus 14 and the injection device is the proper length to be fully inserted to reach the product deposition location 18 in the temporal region. However, as one skilled in the art will appreciate, using longer or short injection devices may cause the dermal access location 16 to vary in the superior or inferior directions. Moreover, not fully inserting the injection device could also cause the dermal access location 16 to vary. Moreover, any combination of the length of the injection device and the amount of insertion could cause the dermal access location 16 to vary in the superior or inferior directions in order to maintain the proper product deposition location 18.

In preferred embodiments, a single injection in the temporal region is performed on both sides of the face. This is done to ensure symmetry in the results. However, in rare occasions, for example when being done to correct an asymmetric facial appearance, a single injection on one side of the face may be performed.

In preferred embodiments, once the injection device has reached the product deposition location 18, the dermal filler is slowly injected in the subdermal plane. As used herein, slowly means anywhere from a rate between 0.05 cc per second and 0.5 cc per second. Preferably, the injection rate is between 0.1 cc per second and 0.2 cc per second.

In preferred embodiments, pressure is applied on an exterior of the skin superficial to the cannula to compress an injection canal created by the cannula and reduce retrograde product flow. While this step is optional, it is recommended to reduce retrograde flow and help localize the injected dermal filler.

During advancement of the injection device, the injection device is preferably lifted against the skin (towards the exterior of the face) to verify that the injection device is located in the subdermal plane and did not change position toward deeper planes. If this lifting technique is employed, the contour of the injection device is readily visible as it advances through the undersurface of the skin, helping to ensure a subdermal position. If a sharp contour of the cannula is not observed, it should be assumed that a deeper plane then the subdermal plane is reached and repositioning of the cannula should be intended.

After the procedure is done, it is recommended that no massaging or manual manipulation is performed or recommended to the patient. However, the patient may be informed about a potential foreign-body sensation and the perception of the increased local surface volume which could last up to seven days after the procedure.

The effects of the inventive methods taught herein were verified on a total of 12 patients (1 male and 11 females) with a mean age of 36.75±6.6 years and a mean body index of 23.04±1.3 kg/m². The injection-related outcome was evaluated utilizing 3D skin vector displacement and surface volume analyses and scored based on five different evaluators. Three months after the treatment, the global esthetic improvement scale rating showed an improved outcome (3.08) when evaluated by the independent observers, very improved outcome when evaluated by the treating physician (4.33), and exceptional improvement when rated by the patient (4.83). Temporal volume improved by 0.36 (P<0.001), lower cheek fullness by 0.47 (P<0.001), and jawline contouring by 0.51 (P<0.001). No major adverse events were reported during the 3-month observational period.

Objective outcome measures have provided evidence that by augmenting the temple using the methods taught herein, the volume of the face can be reduced and the contour of the jawline can be accentuated; this is due to the underlying fascial anatomy and the resulting facial soft tissue lifting effects.

Although the various inventive aspects are herein disclosed in the context of certain preferred embodiments, implementations, and examples, it will be understood by those skilled in the art that the present invention extends beyond the specifically disclosed embodiments to other alternative embodiments and/or uses of the invention and obvious modifications and equivalents thereof. In addition, while a number of variations of the inventive aspects have been shown and described in detail, other modifications, which are within their scope will be readily apparent to those of skill in the art based upon this disclosure. It should be also understood that the scope this disclosure includes the various combinations or sub-combinations of the specific features and aspects of the embodiments disclosed herein, such that the various features, modes of implementation, and aspects of the disclosed subject matter may be combined with or substituted for one another. Thus, it is intended that the scope of the present invention herein disclosed should not be limited by the particular disclosed embodiments or implementations described above, but should be determined only by a fair reading of the claims.

Similarly, this disclosure is not be interpreted as reflecting an intention that any claim require more features than are expressly recited in that claim. Rather, as the following claims reflect, inventive aspects lie in a combination of fewer than all features of any single foregoing disclosed embodiment. Thus, the claims following the Detailed Description are hereby expressly incorporated into this Detailed Description, with each claim standing on its own as a separate embodiment.

Further, all claim terms should be interpreted in their most expansive forms so as to afford the applicant the broadest coverage legally permissible. Although the embodiments have been described with reference to the drawings and specific examples, it will readily be appreciated by those skilled in the art that many modifications and adaptations of the processes, methods and apparatuses described herein are possible without departure from the spirit and scope of the embodiments as claimed herein. Thus, it is to be clearly understood that this description is made only by way of example and not as a limitation on the scope of the embodiments as claimed below.

What is claimed is:

1. A method of enhancing an aesthetic appearance of a human face comprising:
inserting an injection device into a subdermal plane of the human face;
advancing a tip of the injection device in the subdermal plane to a product deposition location in a temporal region from 0.5 cm and 1.5 cm anterior to an apex of a tragus and between 4 cm and 6 cm superior to a horizontal plane through the apex of the tragus; and
injecting a single bolus of between 0.5 cc and 1.5 cc of soft tissue filler material.

2. The method of claim 1, wherein the product deposition location is about 1 cm anterior to the apex of the tragus and about 5 cm superior to the horizontal plane through the apex of the tragus.

3. The method of claim 2, wherein the injection device is inserted in a dermal access location about 1 cm anterior to an apex of a tragus.

4. The method of claim 3, wherein the injection device is advanced in a superior direction from the dermal access location.

5. The method of claim 2, wherein the injection device is a needle and cannula.

6. The method of claim 5, wherein the cannula is a blunt tip cannula.

7. The method of claim 6, wherein the entire length of the cannula is advanced in the subdermal plane.

8. The method of claim 7, wherein during advancement, the cannula is lifted towards an exterior of the face.

9. The method of claim 1, wherein the single bolus of between 0.75 cc and 1.0 cc of soft tissue filler material is injected at a rate between 0.1 cc per second and 0.2 cc per second.

10. The method of claim 1, wherein pressure is applied on an exterior of the skin superficial to the cannula to compress an injection canal created by the cannula and reduce retrograde product flow.

11. The method of claim 1, wherein the soft tissue filler is an HA-based soft tissue filler or a CaHA material.

12. The method of claim 5 wherein the needle is a 21G needle and the cannula is a 22G 50 mm blunt-tip cannula.

13. The method of claim 1, wherein between 0.75 cc and 1.0 cc of soft tissue filler material is injected.

14. The method of claim 1 wherein a single injection in the temporal region is done on each side of the human face.

15. A method of enhancing an aesthetic appearance of a human face comprising:
inserting an injection device into a subdermal plane of the human face at a dermal access location between 0.5 cm and 1.5 cm anterior to an apex of a tragus;
advancing a tip of the injection device in a superior direction in the subdermal plane to a product deposition location in a temporal region between 0.5 cm and 1.5 cm anterior to the apex of the tragus and between 4 cm and 6 cm superior to the dermal access location;
injecting a single bolus of between 0.5 cc and 1.5 cc soft tissue filler material at the injection location.

16. The method of claim 15, wherein the injection device is a needle and blunt tip cannula.

17. The method of claim 15, wherein the soft tissue filler is 1.0 cc of an HA-based soft tissue filler or 0.75 cc of CaHA filler.

18. The method of claim 15, wherein the dermal access location is about 1 cm anterior to the apex of the tragus.

19. The method of claim 15, wherein the product deposition location is about 1 cm anterior to the apex of the tragus and about 5 cm superior to the dermal access location.

20. A method of enhancing an aesthetic appearance of a human face comprising:
inserting a needle and a blunt tip cannula into a subdermal plane of the human face at a dermal access location about 1 cm anterior to an apex of a tragus;
advancing the blunt tip cannula in a superior direction in the subdermal plane to a product deposition location in a temporal region about 1 cm anterior to the apex of the tragus and about 5 cm superior to the dermal access location; and
injecting a single bolus of between 0.75 cc and 1.0 cc HA-based soft tissue filler or a CaHA material at the product deposition location.
